(19) United States
(12) Patent Application Publication
(10) Pub. No.: US 2007/0167723 A1
(43) Pub. Date: Jul. 19, 2007

(54) Optical magnetometer array and method for making and using the same

(75) Inventors: Chang-Min Park, Portland, OR (US); Shriram Ramanathan, Cambridge, MA (US)

Correspondence Address: MORRISON & FOERSTER LLP, 1650 TYSONS BOULEVARD, SUITE 400, MCLEAN, VA, 22102, US

(73) Assignee: Intel Corporation, Santa Clara, CA (US)

(21) Appl. No.: 11/319,792

(22) Filed: Dec. 29, 2005

(51) Int. Cl. G01R 33/02; A61B 5/05

(52) U.S. Cl. 600/409; 324/244.1

(57) ABSTRACT

An embodiment of the invention relates to a device comprising an array of optical magnetometers. The magnetometers comprise se a light source, a container having a chamber filled with an atomic vapor, and a photo detector capable of detecting optical properties of the atomic vapor. The substrate and the array of the magnetometers are designed such that the magnetometers are able to detect weak magnetic fields. Also, the magnetometers are capable of detecting distinct portions of a magnetic field, such as a non-uniform magnetic field, using a single or a plurality of the magnetometers in the array simultaneously. Other embodiment of the invention include methods of making a device that comprises an array of magnetometers and methods of detecting a magnetic field using the device. The device and method can be used in medical diagnostics, such as detecting biomagnetic activities of the human's heart and brain.

### RELATED APPLICATIONS

None.

### FIELD OF INVENTION

The embodiments of the invention relate to a device comprising an array of optical magnetometers, and to a method of making such devices and detection of magnetic fields using such devices. More specifically, the embodiments relate to devices and methods for detecting weak and/or non-uniform magnetic fields, such as biomagnetic fields, using an array of optical magnetometers. The invention transcends several scientific disciplines such as physics, engineering, material science, and medical diagnostics.

### BACKGROUND

Measurement of weak and/or non-uniform magnetic fields is important, or even critical, in many applications, such as geophysical mapping, underground deposit detection, navigation, and medical diagnostics. For example, the detection and measurement of biomagnetic activities, or magnetic fields in living organisms, has becoming increasingly important in disease detection and treatment. Magnetic activities of the heart and brain, often in the pico or femto Tesla (pT or fT) scales, reveal large amount and important information regarding the person's health. However, due to the weakness and particular distributions on or around the human body, biomagnetic activities are difficult to measure and/or analyze.

The superconducting quantum interference device (SQUID) has been used for measuring biomagnetic activities. SQUID consists of two superconductors separated by thin insulating layers to form two parallel Josephson junctions. The device may be configured as a magnetometer to detect weak magnetic fields, such as biomagnetic fields. For example, SQUID has been used to produce magnetocardiogrm (MCG) and magnetoencephalography (MEG) of humans. Squids have also been used to measure the magnetic fields in mouse brains to test whether there might be enough magnetism to attribute their navigational ability to an internal compass.

However, SQUID has at least two drawbacks in its applications. It not only requires cryogenic cooling while being used, but also is large in size, making it inconvenient or impossible to use in many situations. Optical magnetometers are increasingly being explored as an alternative to SQUID in measuring and analyzing weak magnetic fields, including biomagnetic activities.

### BRIEF DESCRIPTION OF THE DRAWINGS

FIG. 1 illustrates an embodiment of the invention that comprises an array of optical magnetometers.

FIG. 2 illustrates a more detailed view of an optical magnetometer.

FIG. 3 illustrates another embodiment of the invention in which a device comprising an array of optical magnetometers is used to detect the magnetic field of a human brain.

### DETAILED DESCRIPTION

An embodiment of the invention relates to a device for detecting a magnetic field. The device comprises an array of optical magnetometers placed on a substrate. Each of the each of the magnetometers comprises a container having a chamber filled with an atomic vapor; a light source capable of transmitting light to the atomic vapor in the chamber; and a photo detector capable of detecting an optical property of the atomic vapor in the chamber. When the device is placed within an external magnetic field, the optical properties of the atomic vapor are changed and detected by the photo detector.

Another embodiment of the invention relates to a method of making a device for detecting a magnetic field. The method comprises providing a substrate and fabricating an array of optical magnetometers on a surface of the substrate. According to the embodiment, each of the optical magnetometers comprises a container having a chamber filled with an atomic vapor, a light source capable of transmitting light to the atomic vapor in the chamber, and a photo detector capable of detecting an optical property of the atomic vapor in the chamber.

A third embodiment of the invention relates to a method of detecting a magnetic field. The method comprises providing a device that comprises a substrate and an array of optical magnetometers placed on the substrate; placing the device within an external magnetic field; and detecting the external magnetic field by simultaneously using at least a portion of the array of optical magnetometers.

As used in the specification and claims, the singular forms “a”, “an” and “the” include plural references unless the context clearly dictates otherwise. For example, the term “an optical magnetometer” may include a plurality of optical magnetometers unless the context clearly dictates otherwise.

An “optical magnetometer” is a magnetometer, or a device for detecting and/or measuring a magnetic field, in which the optical and magnetic properties of paramagnetic atoms are exploited in the detection and/or measurement. An optical magnetometer specifically includes an optically pumped magnetometer (OPM) in which a paramagnetic atom, such as an alkali metal atom, receives a photon and jumps, or is “pumped,” into a higher energy level. The photon is usually provided by a photon emitter, such as a laser. When all such atoms in an enclosed environment receive sufficient photons, the atoms will no longer absorb any photon and reach a relatively stable, or calibrated, state. The photons pass through the atoms in the enclosed environment and other optical properties of the atoms are detected and/or measured by a photo detector at the other side of the photon emitter. When the calibrated atoms are put in an external magnetic field, changes to the energy levels of the atoms are detected by the photo detector, and the magnetic field is detected and/or measured. An optical magnetometer may be a scalar magnetometer or a vector magnetometer. A scalar magnetometer measures the total strength of a magnetic field to which it is subjected, whereas a vector magnetometer has the capability to measure the component of the magnetic field in a particular direction, which may allow the magnetic field's strength, inclination and declination to be uniquely defined.

As used in the embodiments of the invention, “associated with” or “in association with” means that two or more objects are so situated that the desired results or effects are achieved. For example, in an optical magnetometer, a light source, a container, and a photo detector are “associated” with each other when the three components are aligned to perform the basic functions of an optical magnetometer. In other words, the light source emits photons into the container, and the photo detector detects the optical properties of the atomic vapor in the container. As disclosed herein, a number of factors will be considered when designing the optical magnetometer, or associating the light source, the container and the photo detector, including the type and strength of the light source, sizes of the container and its vapor chamber, the type of the atomic vapor, and the type of the photo detector. As disclosed herein, the specific locations and orientations of the light source, the container and the photo detector will be determined based on the specific analysis desired by a person skilled in the art.

As used herein, “dimension” or “dimensions” are the parameters or measurements required to define the shape and/or size, such as height, width, and length, of an object. As used herein, the dimension of a two-dimensional object, such as a rectangle, a polygon, or a circle, is the longest straight-line distance between any two points on the object. Therefore the dimension of a circle is its diameter; a rectangle its diagonal, and a polygon its longest diagonal. The dimension of a three-dimensional object is the longest straight-line distance between any two points on the object. For example, the dimension of a cubical shaped container in an optical magnetometer is the distance between its two opposing vertices. The dimensions used herein are usually measured by centimeters (cm), millimeters (mm), and micrometers (μm), and nanometers (nm).

An “array,” “macroarray” or “microarray” is an intentionally created collection of substances, such as molecules, openings, microcoils, detectors and/or sensors, such as magnetometers, attached to or fabricated on a solid surface, such as glass, plastic, silicon chip or other substrate forming an array. The arrays can be used to measure the expression levels of large numbers of reactions or combinations simultaneously. The substances in the array can be identical or different from each other. The array can assume a variety of formats, e.g., libraries of soluble molecules; libraries of compounds tethered to resin beads, silica chips, or other solid supports. The array could either be a macroarray or a microarray, depending on the size of the pads on the array. A macroarray generally contains pad sizes of about 300 microns or larger and can be easily imaged by gel and blot scanners. A microarray would generally contain pad sizes of less than 300 microns.

An array of optical magnetometers is a collection of optical magnetometers fabricated on a substrate, such as silicon, glass, or polymeric substrate. Each of the optical magnetometers may be associated, corresponded, or otherwise connected to other components such that the signals detected by the magnetometer can be further processed and/or analyzed. The components may be integrated within the substrate or provided by an external source. The array of optical magnetometers may be so arranged that the location of each of the optical magnetometers is precisely calibrated and the magnetic fields being detected by the optical magnetometers are accordingly profiled. Furthermore, in cases where the magnetic fields are not uniformly distributed, as in many biomagnetic activities, the small size of each of the optical magnetometers and the design of the array are such that the magnetic field being experienced by a single optical magnetometer is relatively uniform. Therefore, the optical magnetometer array may be able to detect distinct portions of the magnetic field accurately and, at the same time, provide an accurate profile of the whole magnetic field.

A “substrate” refers to a material or a combination of materials upon and/or within which other or additional materials are formed, attached, or otherwise associated with according to a predetermined fashion. A substrate often provides physical and functional support to the other or additional materials such that, together, they form part or whole of a functional device. A substrate may be a combination of two or more other substrates, which, due to the combination, have become an identifiable new substrate. In the embodiments of the invention, the substrate may comprise silicon, glass, metal, or polymeric materials. In more specific embodiments, the substrate comprises an integrated material, such as an integrated circuit die.

“Solid support” and “support” refer to a material or group of materials having a rigid or semi-rigid surface or surfaces. In some aspects, at least one surface of the solid support will be substantially flat, although in some aspects it may be desirable to physically separate synthesis regions for different molecules with, for example, wells, raised regions, pins, etched trenches, or the like. In certain aspects, the solid support(s) will take the form of beads, resins, gels, microspheres, or other geometric configurations.

As used herein, “magnetic,” “magnetic effect,” and “magnetism” refer to the phenomena by which one material exert an attractive or repulsive force on another material. Although theoretically all materials are influenced to one degree or another by magnetic effect, those skilled in the art understand that magnetic effect or magnetism is only recognized for its detectability under the specific circumstance.

As used herein, a “permanent magnet” is a material that has a magnetic field without relying upon outside influences. Due to their unpaired electron spins, some metals are magnetic when found in their natural states, as ores. These include iron ore (magnetite or lodestone), cobalt, and nickel. A “paramagnetic material” refers to a material that attracts and repels like normal magnets when subject to a magnetic field. Paramagnetic materials include aluminum, barium, platinum, and magnesium. A “ferromagnetic material” is a material that can exhibit a spontaneous magnetization. Ferromagnetism is one of the strongest forms of magnetism and is the basis for all permanent magnets. Ferromagnetic materials include iron, nickel, and cobalt. A “superparamagnetic material” is a magnetic material that exhibits a behavior similar to that of a paramagnetic material at temperatures below the Curie or the Neel temperature.

An “electromagnet” is a type of magnet in which the magnetic field is produced by a flow of electric current. The magnetic field disappears when the current ceases. A simple type of electromagnet is a coiled piece of wire that is electrically connected. An advantage of an electromagnet is that the magnetic field can be rapidly manipulated over a wide range by controlling the electric current. In the embodiments of the invention, ferromagnetic or non-magnetic materials are used to form the electromagnets.

A “microcoil” is a coil, or one or more connected loops, having at least one dimension in the micrometer (μm), or less than 10⁻³ meter (mm), scale. A microcoil usually comprises a thin material wound or gathered around a center or an imaginative center into spiral, helical or other shapes. A microcoil is usually used to produce a defined oscillating magnetic field. A microcoil is defined by the material itself, the shape of the windings, and the separation between each windings. Solenoid type microcoils are multiple spiral wire loops, which may or may not be wrapped around a metallic core. A Solenoid type microcoil produces a magnetic field when an electrical current is passed through it and can create controlled magnetic fields. A Solenoid type microcoil can produce a uniform magnetic field in a predetermined volume of space. A “planar” microcoil is a microcoil with its windings substantially remained in an actual or imaginative plane.

The term “chip” or “microchip” refers to a small device or substrate that comprises components for performing certain functions. A chip includes substrates made from silicon, glass, metal, polymer, or combinations and capable of functioning as a microarray, a macroarray, a microfluidic device, a MEMS, and/or an integrated circuit. A chip may be a microelectronic device made of semiconductor material and having one or more integrated circuits or one or more devices. A “chip” or “microchip” is typically a section of a wafer and made by slicing the wafer. A “chip” or “microchip” may comprise many miniature transistors and other electronic components on a single thin rectangle of silicon, sapphire, germanium, silicon nitride, silicon germanium, or of any other semiconductor material. A microchip can contain dozens, hundreds, or millions of electronic components. In the embodiments of the invention, as discussed herein, microchannels, microfluidic devices, and magnetic tunnel junction sensors can also be integrated into a microchip.

“Micro-Electro-Mechanical Systems (MEMS)” is the integration of mechanical elements, sensors, actuators, and electronics on a common silicon substrate through microfabrication technology. While the electronics are fabricated using integrated circuit (IC) process sequences (e.g., CMOS, Bipolar, or BICMOS processes), the micromechanical components could be fabricated using compatible “micromachining” processes that selectively etch away parts of the silicon wafer or add new structural layers to form the mechanical and electromechanical devices. Microelectronic integrated circuits can be thought of as the “brains” of a system and MEMS augments this decision-making capability with “eyes” and “arms”, to allow Microsystems to sense and control the environment. Sensors gather information from the environment through measuring mechanical, thermal, biological, chemical, optical, and magnetic phenomena. The electronics then process the information derived from the sensors and through some decision making capability direct the actuators to respond by moving, positioning, regulating, pumping, and filtering, thereby controlling the environment for some desired outcome or purpose. Because MEMS devices are manufactured using batch fabrication techniques similar to those used for integrated circuits, unprecedented levels of functionality, reliability, and sophistication can be placed on a small silicon chip at a relatively low cost. In the embodiments of the invention, as discussed herein, MEMS devices are further integrated with microchannels, microfluidic devices, and/or magnetic tunnel junction sensors, such that, together, they perform separation and detection function for biological cells and biomolecules.

“Microprocessor” is a processor on an integrated circuit (IC) chip. The processor may be one or more processor on one or more IC chip. The chip is typically a silicon chip with thousands of electronic components that serves as a central processing unit (CPU) of a computer or a computing device.

A “nanomaterial” as used herein refers to a structure, a device or a system having a dimension at the atomic, molecular or macromolecular levels, in the length scale of approximately 1-1000 nanometer (nm) range. Preferably, a nanomaterial has properties and functions because of the size and can be manipulated and controlled on the atomic level.

One embodiment of the invention relates to a device for detection and measurement of magnetic fields using an array of optical magnetometers. The device comprises a substrate and an array of optical magnetometers placed on the substrate. According to the embodiment, at least one of the magnetometers comprises a container having a chamber filled with an atomic vapor, and the atomic vapor has an optical property capable of being changed by the presence of an external magnetic field across the chamber.

In one embodiment, the device further comprises a light source capable of transmitting light to the atomic vapor in the chamber. Specifically, the light source may be comprised by the magnetometer. Also, the device may further comprise a photo detector capable of detecting an optical property of the atomic vapor. Specifically, the photo detector may be comprised by the magnetometer. The external magnetic field, or the magnetic field to be measured, may be oriented at different angles with the orientation of the light, from parallel, to 45°, to perpendicular with each other. In a specific embodiment, the external magnetic field is parallel to the light.

The embodiments of the invention thus encompass devices that contain an array of optically pumped magnetometers. As disclosed herein, an optical pumped magnetometer uses an ionizing light beam, e.g., from a laser, to manipulate one of several elements from a specific group of atoms within a sample volume for the purpose of observing their reaction to external magnetic forces. By manipulating and monitoring the nuclei of any one of the atoms, usually alkali metals, measurements can be made of magnetic forces.

Alkali metals are very reactive to certain external forces and will easily lose an electron such as when a photon or ionizing light energy is applied. The term used for applying constant energy from light for the purpose of elevating an electron to an outer orbit, is “optically pumped.” However, magnetic forces have a stabilizing effect on alkali metals that have lost an electron and tend to force any losing electron back to its stable neutral state, thus counteracting the ionizing light energy or optically pumped energy. The moving of the electrons and the associated changes in energy levels, as demonstrated by light emissions, can be monitored and measured within a confined volume of anatomic vapor. For example, stronger magnetic fields will stabilize electrons at a faster rate than weaker fields. Energy gained by the electron when forced from its outer orbit is lost when it is forced back to its neutral state by a repelling energy, such as a magnetic force. By monitoring the gain and loss of energy in a volume of alkali gas one can relate, proportionately, magnetic field strengths.

In the embodiment of the invention, an optical magnetometer or, more specifically, an optically pumped magnetometer, an alkali vapor, such as cesium or potassium is sealed within a container having a chamber where an ionizing light can be emitted or “pumped” into, sometimes through various optical filters. The ionizing light energizes the molecules in the chamber and ejects electrons from the outermost orbit of individual electrons. An external magnetic field near the chamber can force the electrons back to their stable state. During this process the loss of energy due to the electrons dropping down to their stable state must be released and is given off as a spark of light. A photo detector, a device that measures light intensity, at the other end of the chamber measures the amount of light given off. Greater light intensity means that a strong magnetic field is quickly forcing electrons back to a normal state within the sample volume. Weaker magnetic fields will not cause the electrons to return to normal as rapidly, thus producing less light in the sample volume. The rate at which electrons revert back to normal is proportional to the magnetic field strength and thus provides a measurable value.

In one embodiment, the device further comprises a magnet capable of producing an oscillating magnetic field across the chamber. In a specific embodiment, the magnet is an electromagnet, such as in the form of a microcoil, capable of producing an oscillating magnetic field. As disclosed herein, as atoms spin, magnetic moments are associated with the atoms. If another, oscillating, magnetic field is present, the atomic spins may precess. This precession may help enhance the sensitivity of an optical magnetometer by creating a magnetic resonance effect of the atoms using the oscillating magnetic field. The orientations of the oscillating magnetic field, the external magnetic field, and the light going in and out of the chamber can be arranged in a variety of manners according to specific applications and designs in order to achieve more sensitive and/or accurate results. For example, in certain situations, the oscillating magnetic field is oriented to be perpendicular to the external magnetic field, or the magnetic field to be measured. In this orientation, both magnetic fields may be oriented at about a 45° angle to the direction of the light being transmitted into the chamber. Thus, in a specific embodiment of the invention, the oscillating magnetic field is oriented at about 45° with respect to the light transmitted to the atomic vapor in the chamber.

In another embodiment of the invention, the substrate comprises components capable of processing or analyzing signals from the photo detector. Further, the components may comprise one or more of a control, display, amplifier, microprocessor, MEMS, and integrated circuit. According to the embodiment, the substrate not only provides support and a platform for the array of magnetometers, but also provides necessary electrical and/or mechanical components that facilitate the functions of the optical magnetometers and process data detected and collected by the magnetometers. In this regard, the substrate may comprise silicon and/or glass based integrated circuits to perform the functions.

Silicon is a suitable material for forming and/or attaching optical magnetometers coupled with microelectronics or other microelectromechanical systems (MEMS). It also has good stiffness, allowing the formation of fairly rigid microstructures, which can be useful for dimensional stability. In a specific embodiment of the invention, the substrate comprises an integrated circuit (IC), a packaged integrated circuit, and/or an integrated circuit die. For example, the substrate may be a packaged integrated circuit that comprises a microprocessor, a network processor, or other processing device. The substrate may be constructed using, for example, a Controlled Collapse Chip Connection (or “C4”) assembly technique, wherein a plurality of leads, or bond pads are internally electrically connected by an array of connection elements (e.g., solder bumps, columns).

Specific materials useful as the substrate also include, but not limited to, polystyrene, polydimethylsiloxane (PDMS), glass, chemically functionalized glass, polymer-coated glass, nitrocellulose coated glass, uncoated glass, quartz, natural hydrogel, synthetic hydrogel, plastics, metals, and ceramics. The substrate may comprise any platform or device currently used for carrying out medical diagnostics. Thus, the substrate may comprise a microarray or a macroarray, a multi-well plate, a microfluidic device, or a combination thereof.

In another embodiment, the substrate comprises circuitry that is capable of amplifying, processing, and/or analyzing the signals detected by the photo detector. Any suitable conventional circuits may be used and integrated into the substrate for amplifying and/or processing, including filtering, the signals. The integrated circuitry may be able to generate magnetic field profiles or maps independently or connected to an external device for generating the profiles or maps.

According to another embodiment of the invention, the magnetometer is a scalar or vector magnetometer. As disclosed herein, a scalar magnetometer measures the total strength of a magnetic field to which it is subjected, whereas a vector magnetometer has the capability to measure the component of the magnetic field in a particular direction. In another embodiment, the array of optical magnetometers on the device is capable of providing data for generating a two-dimensional or three-dimensional mapping of the external magnetic field.

In one embodiment of the invention, the array of optical magnetometers is placed on a surface of the substrate. According to this embodiment, the array of optical magnetometers may themselves form a surface according to a pre-determined manner. The embodiment allows the device to be used in detecting and measuring biomagnetic activities such as human heart and brain magnetic activities by aligning the array of optical magnetometers according to the contours of the human body.

In one embodiment, the array of optical magnetometers is capable of being placed on or near a human's chest and that the device can detect magnetic fields of the human's heart. Further, the surface of the substrate is flat, curved, or a combination thereof and the magnetometers are capable of covering at least a portion of a human's chest. Thus, according to the embodiment, the substrate and the array of optical magnetometers are so designed such that the magnetometers are capable of closely covering part or whole of a human's chest. The embodiment allows a more sensitive, consistent, and/or accurate detection and measurement of the heart's magnetic activities.

In another embodiment, the array of optical magnetometers is capable of being placed on or near a human's head and that the device can detect magnetic fields of the human's brain. Further, the surface of the substrate has a curved or helmet-like shape and the magnetometers are capable of covering at least a portion of a human's head. Thus, according to the embodiment, the substrate and the array of optical magnetometers are so designed such that the magnetometers are capable of closely covering part or whole of a human's head. The embodiment allows a more sensitive, consistent, and/or accurate detection and measurement of the brain's magnetic activities.

In the embodiments of the invention, an optical magnetometer may further comprise components that facilitate and/or enhance the functions of the magnetometer and the detection and measurement of magnetic fields. Thus, at least a portion of the optical magnetometers in the array comprise a polarizer, a quarter plate, a filter, or a combination thereof. These components help to prepare the light from the light source before entering into the chamber so that appropriate “pumping” of the atomic vapor under specific circumstances can be made.

In one embodiment of the invention, the container comprises silicon, glass, a polymer, or a combination thereof. For example, the container may comprise a combination of silicon and glass. In another embodiment, the container and the chamber independently has a cylindrical, cubic, or cuboidal shape. The container has an overall dimension of from about 0.1 mm to about 10 cm, or more specifically, an overall dimension of from about 1.0 mm to about 5.0 cm. At the same time, the chamber has a volume of from about 100 (μm)³ to about 1.0 (cm)³, or more specifically, a volume of from about 1000 (μm)³ to about 10 (mm)³.

The container and the chamber are an important component in an optical magnetometer. The materials and design for the container and chamber are selected and made according to the disclosures herein or conventional technologies. According to one embodiment, a combination of silicon and glass is used to fabricate the container and chamber. Glass allows light to be transmitted in and out of the chamber; and silicon allows an easier fabrication of the container and its integration with other components and devices. Although the container and chamber often have cylindrical, cubical or cuboidal shapes, other shapes and configurations may also facilitate the functions in specific circumstances. Further, the shape of the chamber can be independent from the shape of the chamber as designs require. According to the embodiments of the invention, the container and the chamber have a wide range of sizes, although miniaturized containers are more suitable for fabricating devices comprising an array of optical magnetometers.

In the embodiments of the invention, a variety of materials may be used as the atomic vapor in the chamber. According to one embodiment, the atomic vapor in the chamber comprises lithium (Li), sodium (Na), potassium (K), rubidium (Rb), cesium (Cs), and francium (Fr), or a combination thereof. According to another embodiment, the chamber is also filed with a buffer gas. The buffer gas may comprise nitrogen, helium, neon, argon, krypton, xenon, radon, or a combination thereof.

The atomic vapor and the buffer gas can be filled into the chamber according to methods disclosed herein. The amount of the vapor is determined by the specific requirement of the optical magnetometer and may be controlled by controlling the temperature of the chamber. For example, a special resistance wire may be used to form a heater wrapped around the outside of the container. In spite of the fact that the heater is constructed in this way it still must be driven with an AC wave form to prevent offsetting the magnetic field to be measured. The chamber's temperature may be monitored by a thermistor so that it can be regulated.

The buffer gas is used, in part, to keep the atoms in the vapor from moving too far too quickly. Any atom that hits the wall of the chamber will have its state randomized and will not add to the signal. Any that cross from one side of the chamber to the other side where the sense of polarization is reversed will actually subtract from the signal. In such circumstances, a buffer gas, usually an inert gas is necessary.

According to one embodiment of the invention, the light source comprises a laser. The laser may be a vertical-cavity surface-emitting laser (VCSEL), an actively frequency-stabilized diode laser, or other types of laser. In one embodiment, the light source is capable of transmitting a circularly polarized laser beam to the atomic vapors. In another embodiment, the light source comprises a beam splitter. The photo detector used in the optical magnetometers may be a photodiode.

According to the embodiments of the invention, the light source of an optical magnetometer may be either a source of light, such as a laser lamp, or a light from a source, which may be internal or external of the magnetometer. For example, the magnetometer may itself comprise a laser source or share, e.g., by way of a splitter, a laser beam with a number of other magnetometers. In the latter case, a single laser source may provide light to a part or whole of the array of optical magnetometers.

According to one embodiment of the invention, the light needed to move the electrons from one orbit to the next should have a frequency that corresponds to the energy needed for the specific atoms. According to the embodiment, alkali metal discharge lamps may be used as the light source. The lamp may make the desired light along with other undesirable light which must be removed with a filter. The lamp may be powered inductively because alkali metals are chemically reactive that it may not be possible to use electrodes inside the lamp. The lamp may be surrounded by an induction coil driven with a frequency determined for the specific application.

A laser, as used herein, is an optical source that emits photons in a coherent beam. Laser light is typically near-monochromatic, i.e. consisting of a single wavelength or hue, and emitted in a narrow beam. This is in contrast to common light sources, such as the incandescent light bulb, which emit incoherent photons in almost all directions, usually over a wide spectrum of wavelengths. Light is an electromagnetic wave. In all electromagnetic waves there is an electrostatic vector at right angles to the direction of propagation. In linearly polarized light this vector can be thought of as growing in the positive direction vertically then shrinking to zero and growing in the negative direction as the light travels along. In circularly polarized light the length of this vector is constant but it rotates like the threads on a screw as it travels along.

There can be two types of circular polarization, left and right handed, depending on the direction the field rotates as it propagates. If a photon of laser from the light source has exactly the right amount of energy, an electron of an atom in the chamber can absorb it, moving the electron up to a higher orbit. With circularly polarized laser beam, this works much better if the direction of spin of the electron matches the direction of polarization. If a circularly polarized laser beam is transmitted into the chamber. The electrons that have a spin that matches the polarized light's direction will absorb the light and be kicked up to a higher orbit. However, when in this higher orbit the electrons are not stable and will immediately decay or fall back down releasing energy as light, and their spin direction becomes randomized in the process. The light the electrons give off when they fall is not aligned to the path of the absorbed light. For this reason the light passing through the chamber will be dimmed slightly by the electrons absorbing it. Because the electron's spin axis is random when it falls back down, there is a chance it will be aligned so that the light cannot kick it back to the higher orbit again. Over time all the electrons will eventually land with their spin axes in a manner that will not allow them to absorb the light. When this happens the light passes through the absorption cell and impinges on the photo detector, which detects and/or measures the optical events.

According to the embodiments of the invention, data from optical magnetometers can be collected in one of three ways. One method is to obtain data in a search mode where only data values are shown on the device, such as on a control panel, as the device is moved through a magnetic field, but no positional data are recorded. Another method is collecting data using a sequential numbering system which may automatically advance each time the operator wants a reading to be recorded. This method works best if each increasing numeric value can be tied to some type of location. Another method is to establish a grid system having lines and positions over the area of magnetic field to be surveyed. The lines are preprogrammed into the optical magnetometer to match the grid coordinate system and positions are obtained by starting and stopping constant data recording at the ends of each line. An internal program may be used to automatically post a grid coordinate to each data position point. This data collection method may require, and assume, that a constant moving pace is maintained between the start and finish of each line.

In one embodiment of the invention, the presence of an external magnetic field across each of at least a portion of the chambers is capable of changing the optical properties of the atomic vapors in each of the at least a portion of the chambers. According to the embodiment, the device is capable of measuring an external magnetic field using part or whole of the array of optical magnetometers simultaneously. The embodiment is useful in situations where the external magnetic field is not uniformly distributed or contains distinct areas. In the embodiment, especially when each of the optical magnetometers is sufficiently small such that the magnetic field experienced by the individual magnetometer is substantially uniform, the device is capable of detecting a more detailed profile of the external magnetic field.

In another embodiment, the external magnetic field has a magnetic flux density of from about 10⁻¹⁶ Tesla (T) to about 10⁻⁹ T. The embodiment allows the device to measure very weak magnetic fields, such as biomagnetic fields, e.g., magnetic fields found in human hearts and brains. As disclosed herein, the device may be designed in such a way that the magnetometers can be closely placed to the chest or head of a human to facilitate such measurements.

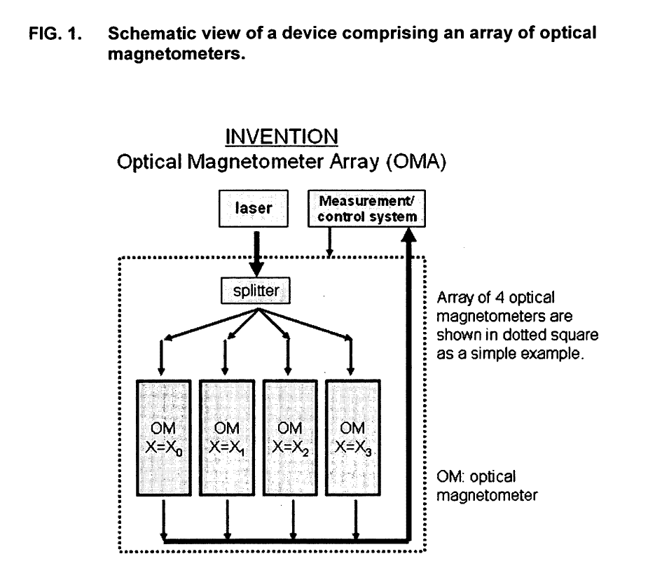

FIG. 1 illustrates an embodiment of the invention where an array of optical magnetometers is used in a single device. As shown, the device comprises an array of optical magnetometers (four are shown in the figure). Each of the magnetometers may be the same or different from each other depending on the specific design. The magnetometers may use a single laser source with a beam splitter directing light into each magnetometer. The device also comprises a measurement and control system, including a photo detector (not shown), that facilitates the application of the device and the processing and/or analyzing the data collected by the magnetometers.

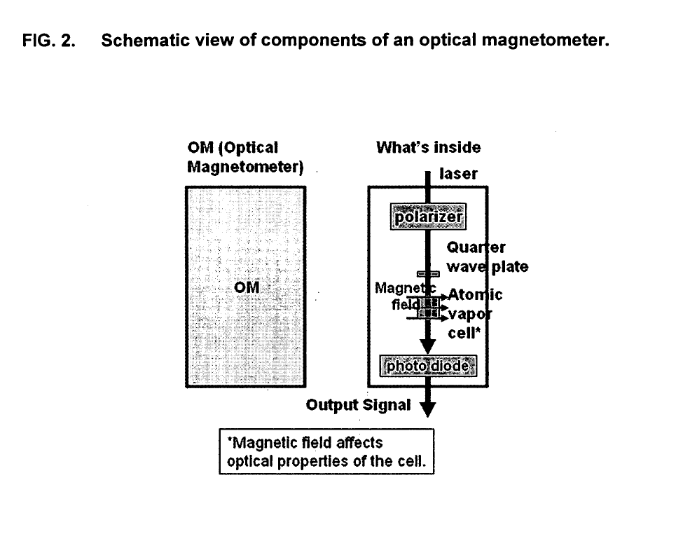

FIG. 2 illustrates a more detailed view of an optical magnetometer according to one embodiment of the invention. As shown, the optical magnetometer comprises a polarizer, a quarter wave plate, an atomic vapor cell or container, and a photodiode. The polarizer and quarter wave plate help to prepare light from a laser source before the light enters the atomic vapor container. The photodiode detects optical signals from the atomic vapor container before, during and after the container is immersed within an external magnetic field. The photodiode transforms the optical signals into electrical signals, which is output for further processing and analyzing.

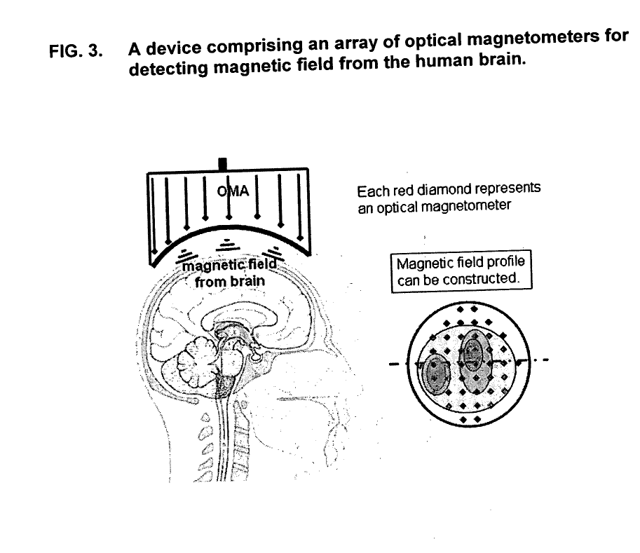

FIG. 3 illustrates another embodiment of the invention in which a device comprising an array of optical magnetometers is used for detecting magnetic filed of a human brain. As shown, a surface of the substrate is configured to form a helmet-like curve. An array of optical magnetometers is formed on the surface. The magnetometers are able to cover at least part of the head and have close relations with the head in a relatively uniform manner. The device is capable of producing data to construct a profile of the brain's magnetic field.

Another embodiment of the invention relates to a method of making a device that comprises an array of optical magnetometers. The method comprises providing a substrate and fabricating an array of optical magnetometers on a surface of the substrate. According to the embodiment, at least one of the optical magnetometers comprises a container having a chamber filled with an atomic vapor, and the atomic vapor has an optical property capable of being changed by the presence of an external magnetic field across the chamber.

In an embodiment, the device further comprises a light source capable of transmitting light to the atomic vapor in the chamber. Specifically, the light source may be comprised by the magnetometer. Also, the device may further comprise a photo detector capable of detecting an optical property of the atomic vapor. Specifically, the photo detector may be comprised by the magnetometer.

In one embodiment, the device further comprises a magnet capable of producing an oscillating magnetic field across at least one of the chambers. In a specific embodiment, the oscillating magnetic field is oriented at about 45° with respect to the light transmitted to the atomic vapor in the chamber. In a specific embodiment, the magnet is an electromagnet, such as in the form of a microcoil, capable of producing an oscillating magnetic field. The magnet can be fabricated either on the substrate or on an optical magnetometer itself.

In another embodiment of the invention, the substrate comprises components capable of processing or analyzing signals from the photo detector. Further, the components may comprise one or more of a control, display, amplifier, microprocessor, MEMS, and integrated circuit. According to the embodiment, the substrate not only provides support and a platform for the array of magnetometers, but also provides necessary electrical and/or mechanical components that facilitate the functions of the optical magnetometers and process data detected and collected by the magnetometers. In this regard, the substrate may comprise silicon and/or glass based integrated circuits to perform the functions.

Silicon is a suitable material for forming and/or attaching optical magnetometers coupled with microelectronics or other microelectromechanical systems (MEMS). It also has good stiffness, allowing the formation of fairly rigid microstructures, which can be useful for dimensional stability. In a specific embodiment of the invention, the substrate comprises an integrated circuit (IC), a packaged integrated circuit, and/or an integrated circuit die. For example, the substrate may be a packaged integrated circuit that comprises a microprocessor, a network processor, or other processing device. The substrate may be constructed using, for example, a Controlled Collapse Chip Connection (or “C4”) assembly technique, wherein a plurality of leads, or bond pads are internally electrically connected by an array of connection elements (e.g., solder bumps, columns).

Specific materials useful as the substrate also include, but not limited to, polystyrene, polydimethylsiloxane (PDMS), glass, chemically functionalized glass, polymer-coated glass, nitrocellulose coated glass, uncoated glass, quartz, natural hydrogel, synthetic hydrogel, plastics, metals, and ceramics. The substrate may comprise any platform or device currently used for carrying out medical diagnostics. Thus, the substrate may comprise a microarray or a macroarray, a multi-well plate, a microfluidic device, or a combination thereof.

In another embodiment, the substrate comprises circuitry that is capable of amplifying, processing, and/or analyzing the signals detected by the photo detectors. Any suitable conventional circuits may be used and integrated into the substrate for amplifying and/or processing, including filtering, the signals. The integrated circuitry may be able to generate magnetic field profiles or maps independently or connected to an external device for generating the profiles or maps.

In one embodiment of the invention, the array of optical magnetometers is placed on a surface of the substrate. According to this embodiment, the array of optical magnetometers may themselves form a surface according to a pre-determined manner. The embodiment allows the device to be used in detecting and measuring biomagnetic activities such as human heart and brain magnetic activities by aligning the array of optical magnetometers according to the contours of the human body.

In one embodiment, the array of optical magnetometers is capable of being placed on or near a human's chest and that the device can detect magnetic fields of the human's heart. In another embodiment, the array of optical magnetometers is capable of being placed on or near a human's head and that the device can detect magnetic fields of the human's brain.

In the embodiments of the invention, an optical magnetometer may further comprise components that facilitate and/or enhance the functions of the magnetometer and the detection and measurement of magnetic fields. Thus, each of at least a portion of the optical magnetometers in the array comprises a polarizer, a quarter plate, a filter, or a combination thereof. These components help to prepare the light from the light source before entering into the chamber so that appropriate “pumping” of the atomic vapor under specific circumstances can be made.

In one embodiment of the invention, the container comprises silicon, glass, a polymer, or a combination thereof. For example, the container may comprise a combination of silicon and glass. In another embodiment, the container and the chamber independently have a cylindrical, cubic, or cuboidal shape.

The container and the chamber are an important component in an optical magnetometer. The materials and design for the container and chamber are selected and made according to the disclosures herein or conventional technologies. According to one embodiment, a combination of silicon and glass is used to fabricate the container and chamber. Glass allows light to be transmitted in and out of the chamber; and silicon allows an easier fabrication of the container and its integration with other components and devices. Although the container and chamber often have cylindrical, cubical or cuboidal shapes, other shapes and configurations may also facilitate the functions in specific circumstances. Further, the shape of the chamber can be independent from the shape of the chamber as designs require. According to the embodiments of the invention, the container and the chamber have a wide range of sizes, although miniaturized containers are more suitable for fabricating devices comprising an array of optical magnetometers.

In the embodiments of the invention, a variety of materials may be used as the atomic vapor in the chamber. According to one embodiment, the atomic vapor the chambers independently comprise lithium (Li), sodium (Na), potassium (K), rubidium (Rb), cesium (Cs), and francium (Fr), or a combination thereof. According to another embodiment, each of at least a portion of the chambers is also filed with a buffer gas. The atomic vapor and the buffer gas can be filled into the chamber according to methods disclosed herein. The amount of the vapor is determined by the specific requirement of the optical magnetometer and may be controlled by controlling the temperature of the chamber.

According to one embodiment of the invention, the light source comprises a laser. The laser may be a vertical-cavity surface-emitting laser (VCSEL), an actively frequency-stabilized diode laser, or other types of laser. In one embodiment, the light source is capable of transmitting a circularly polarized laser beam to the atomic vapors. In another embodiment, the light source comprises a beam splitter. The photo detectors used in the optical magnetometers may be photodiodes.

According to the embodiments of the invention, the light source of an optical magnetometer may be either a source of light, such as a laser lamp, or a light from a source, which may be internal or external of the magnetometer. For example, the magnetometer may itself comprise a laser source or share, e.g., by way of a splitter, a laser beam with a number of other magnetometers. In the latter case, a single laser source may provide light to a part or whole of the array of optical magnetometers.

In one embodiment of the invention, the presence of an external magnetic field across each of at least a portion of the chambers is capable of changing the optical properties of the atomic vapors in each of the at least a portion of the chambers. According to the embodiment, the device is capable of measuring an external magnetic field using part or whole of the array of optical magnetometers simultaneously. The embodiment is useful in situations where the external magnetic field is not uniformly distributed or contains distinct areas. In the embodiment, especially when each of the optical magnetometers is sufficiently small such that the magnetic field experienced by the individual magnetometer is substantially uniform, the device is capable of detecting a more detailed profile of the external magnetic field.

In another embodiment, the external magnetic field has a magnetic flux density of from about 10⁻¹⁶ Tesla (T) to about 10⁻⁹ T. The embodiment allows the device to measure very weak magnetic fields, such as biomagnetic fields, e.g., magnetic fields found in human hearts and brains. As disclosed herein, the device may be designed in such a way that the magnetometers can be closely placed to the chest or head of a human to facilitate such measurements.

The devices of the embodiments of the invention may be formed by any suitable means of manufacture, including semiconductor manufacturing methods, microforming processes, molding methods, material deposition methods, etc., or any suitable combination of such methods. In certain embodiments, one or more of the substrate, the magnetometers, the magnet, such as a microcoil, and the circuitries on the substrate may be formed via semiconductor manufacturing methods on a semiconductor substrate. Thin film coatings may be selectively deposited on portions of the substrate surface. Examples of suitable deposition techniques include vacuum sputtering, electron beam deposition, solution deposition, and chemical vapor deposition. The coatings may perform a variety of functions. Conductive coatings may be used to form the microcoils. Coatings may be used to provide a physical barrier on the surface, e.g. to retain fluid at specific sites on the surface.

According to the embodiments of the invention, optical magnetometers, including the atomic vapor containers and chambers, the light sources and the photo detectors, can be fabricated on or within the substrate using a number of techniques, including etching, bonding, annealing, adhering/seeding, lithography, molding, and printing. Physical vapor deposition (PVD) and chemical vapor deposition (CVD) can also be used. In one embodiment, optical magnetometers are fabricated on an oxidized silicon substrate by electroplating metals inside a deep photoresist mold and then passivated using an epoxy based resist.

According to one embodiment of the invention, optical magnetometers, including components for the light source, the container and the photo detector, can be fabricated on the substrate using the anodic bonding method. Anodic bonding, also referred to as field assisted glass-silicon sealing, is a process which permits the sealing of silicon to glass below the softening point of the glass. The two surfaces to be bonded together have a small surface roughness, usually less than about 0.1 μm to allow the surfaces to match closely. The pieces to be bonded are assembled and heated in room atmosphere at temperatures between about 400 to about 500° C. A DC power supply is connected to the assembly such that the silicon is positive with respect the glass. When a voltage on the order of a few hundred volts is applied across the assembly the glass seals to the metal.

In one embodiment of the invention, the container can be made according to the following method. A polished double-sided silicon wafer was patterned using photolithography method in a clean room and etched in either KOH or reactive ion to produce a roughly square hole with dimensions ranging from tens of micrometers to a few millimeters. The silicon wafer was then diced into individual chips with dimensions of about 0.5 to 2.0 centimeters. The silicon chips were then anodically bonded to similarly-sized glass. The combined piece now has a squared shaped cavity. The cavity was then filled with potassium and a buffer gas and sealed by anodically bonding a second glass chip to the top the cavity, which became a sealed chamber. The filling of the potassium and buffer gas can be accomplished by chemical reactions or injection methods. The final container consisted of a three-layered bonded structure with optically transparent glass windows on either side of the potassium-containing chamber.

In the embodiments of the invention, optical magnetometers can also be made by using soft lithography method with suitable materials, such as silicon and polydimethylsiloxane (PDMS). With these techniques it is possible to generate patterns with critical dimensions as small as 30 nm. These techniques use transparent, elastomeric PDMS “stamps” with patterned relief on the surface to generate features. The stamps can be prepared by casting prepolymers against masters patterned by conventional lithographic techniques, as well as against other masters of interest. Several different techniques are known collectively as soft lithography. They are as described below:

Near-Field Phase Shift Lithography.

A transparent PDMS phase mask with relief on its surface is placed in conformal contact with a layer of photoresist. Light passing through the stamp is modulated in the near-field. Features with dimensions between 40 and 100 nm are produced in photoresist at each phase edge.

Replica Molding.

A PDMS stamp is cast against a conventionally patterned master. Polyurethane is then molded against the secondary PDMS master. In this way, multiple copies can be made without damaging the original master. The technique can replicate features as small as 30 nm.

Micromolding in Capillaries (MIMIC).

Continuous channels are formed when a PDMS stamp is brought into conformal contact with a solid substrate. Capillary action fills the channels with a polymer precursor. The polymer is cured and the stamp is removed. MIMIC is able to generate features down to 1 μm in size.

Microtransfer Molding ((TM).

A PDMS stamp is filled with a prepolymer or ceramic precursor and placed on a substrate. The material is cured and the stamp is removed. The technique generates features as small as 250 nm and is able to generate multilayer systems.

Solvent-assisted Microcontact Molding (SAMIM).

A small amount of solvent is spread on a patterned PDMS stamp and the stamp is placed on a polymer, such as photoresist. The solvent swells the polymer and causes it to expand to fill the surface relief of the stamp. Features as small as 60 nm have been produced.

Microcontact Printing ((CP). An “ink” of alkanethiols is spread on a patterned PDMS stamp. The stamp is then brought into contact with the substrate, which can range from coinage metals to oxide layers. The thiol ink is transferred to the substrate where it forms a self-assembled monolayer that can act as a resist against etching. Features as small as 300 nm have been made in this way.

Techniques used in other groups include micromachining of silicon for microelectromechanical systems, and embossing of thermoplastic with patterned quartz. Unlike conventional lithography, these techniques are able to generate features on both curved and reflective substrates and rapidly pattern large areas. A variety of materials could be patterned using the above techniques, including metals and polymers. The methods complement and extend existing nanolithographic techniques and provide new routes to high-quality patterns and structures with feature sizes of about 30 nm.

Another embodiment of the invention relates to a method of detecting and measuring a magnetic field using a device comprising an array of optical magnetometers. The method comprises providing a device that comprises a substrate and an array of optical magnetometers placed on the substrate; placing the device within an external magnetic field; and detecting the external magnetic field by simultaneously using at least a portion of the array of optical magnetometers. In a specific embodiment, each of the optical magnetometers comprises a container having a chamber filled with an atomic vapor; a light source capable of transmitting light to the atomic vapor in the chamber; and a photo detector capable of detecting an optical property of the atomic vapor in the chamber. In another specific embodiment, an oscillating magnetic field across at least one of the chambers is provided.

In one embodiment, the placing of the device within an external magnetic field comprises adjusting the location and orientation of the device. As disclosed herein, the external magnetic field, or the magnetic field to be measured, may be oriented at different angles with the orientation of the light, from parallel, to 45°, to perpendicular with each other. Adjustment of the device may be made according to the specific application and design of the device in order to achieve more sensitive and/or accurate measurements.

The embodiment allows a more effective use of the device. As disclosed herein, the embodiments of the invention have faster measuring cycles which can be obtained as often as 0.1 second and greater accuracy in measuring magnetic field strength. The device, however, should be handled with care when used in order to avoid any “dead zone” fields of view in the device due to the required configuration of internal components. Properly positioning or orienting the device for the specific location or latitude (a relationship which determines angles of magnetic fields at a latitude) will reduce the “dead zone” effect, and allow for an efficient measurement. Establishing proper device angles can be obtained based on knowledge and/or available charts, tables or other data.

In one embodiment of the invention, the detecting of the external magnetic field comprises detecting each distinct portion of the external magnetic field using a distinct magnetometer. According to the embodiment, the method and device are capable of detecting and/or measuring a distinct portion of the external magnetic field using an individual, part or whole of the optical magnetometers in the array simultaneously. The embodiment is useful in situations where the external magnetic field is not uniformly distributed or contains distinct areas. In the embodiment, especially when each of the optical magnetometers is sufficiently small such that the magnetic field experienced by the individual magnetometer is substantially uniform, the method is capable of detecting a more detailed profile of the external magnetic field.

According to another embodiment of the invention, each of at least a portion of the magnetometers is independently a scalar or vector magnetometer. As disclosed herein, a scalar magnetometer measures the total strength of a magnetic field to which it is subjected, whereas a vector magnetometer has the capability to measure the component of the magnetic field in a particular direction. In another embodiment, the array of optical magnetometers on the device is capable of providing data for generating a two-dimensional or three-dimensional mapping of the external magnetic field.

In another embodiment, the method of detecting a magnetic field is carried out in a magnetically shield environment. The method is especially useful in medical diagnostics where the detection of very weak biomagnetic fields is made. A magnetically shielded environment may be necessary when using the device to detect and measure minute biomagnetic signals radiated from various parts of the human body, including the heart and the brain. A magnetically shielded environment, such as a magnetically shielded room, improves the strength of the useful signal by excluding the background magnetism and other spurious magnetic fields. A magnetically shielded environment can be an enclosure with a shell comprising layers of high permeability metals that are also good electrical conductors. This attenuates, or absorbs, the spurious magnetic and electrical fields emanating from numerous sources in buildings such as hospitals.

In a specific embodiment, the placing of the device within an external magnetic field comprises placing the device on or near a human's chest. The embodiment further comprises generating a magnetocardiogram (MCG) of the human. Thus the embodiment encompasses a method of medical diagnostics, specifically, producing data and generating a magnetocardiogram (MCG) of a human.

Magnetocardiography is the measurement of magnetic fields emitted by the human heart from small currents by electrically active cells of the heart muscle. The measurement of these fields over the torso provides information which is complementary to that provided by electrocardiography (ECG), used especially in diagnosing abnormalities of heart function. An MCG could provide much information about the heart's electric activity as could the ECG and has many potential clinical applications. For instance, according to the present understanding, with the combined use of the ECG and the MCG, called electromagnetocardiogram (EMCG), in some cardiac diseases the number of incorrectly diagnosed patients can be decreased by one half of that when using only the ECG is used.

According to the embodiment, the design of the device with an array of optical magnetometers capable of being closely placed on or near the chest, detection and MCG can be made of the magnetic heart vector as well as of the normal component of the magnetic field of the heart around the thorax.

In another specific embodiment, the placing of the device within an external magnetic field comprises placing the device on or near a human's head. The embodiment further comprises generating a magnetoencephalogram (MEG) of the human. Thus the embodiment encompasses a method of medical diagnostics, specifically, producing data and generating a magnetocardiogram (MCG) of a human.

Magnetoencephalography (MEG) is the measurement of magnetic fields emitted by the human brain from small currents by electrically active cells of the brain. MEG is a noninvasive, non-hazardous technology for functional brain mapping, providing spatial good discrimination and temporal resolution. It can localize and characterize the electrical activity of the central nervous system by measuring the associated magnetic fields emanating from the brain. The information provided by MEG is different from that provided by Computed Tomography (CT) or Magnetic Resonance (MR) imaging. Unlike the latter two which provide structural/anatomical information, MEG provides functional mapping information. MEG is a functional imaging capability complementary to the anatomical imaging capabilities of MRI, CT.

According to the embodiment, the method and device can be used to measure the activity of the brain in real time. The brain can be observed “in action” rather than just viewing a still image. MEG data obtained by the method and device can be used to identify both normal and abnormal functions of brain structures which are anatomically so crisply seen in the static MRI scans.

Further, the method and device is able to not only locate the sources of evoked responses, but also record signals quickly over the whole cortex by using an array of optical magnetometers. This will lead the focus to studies of spontaneous activity and its changes during various tasks.

The characteristics of some of the embodiments of the invention are illustrated in the Figures and examples, which are intended to be merely exemplary of the invention. This application discloses several numerical range limitations that support any range within the disclosed numerical ranges even though a precise range limitation is not stated verbatim in the specification because the embodiments of the invention could be practiced throughout the disclosed numerical ranges. Finally, the entire disclosure of the patents and publications referred in this application, if any, are hereby incorporated herein in entirety by reference.

1. A device comprising a substrate and an array of optical magnetometers placed on the substrate, wherein at least one of the magnetometers comprises a container having a chamber filled with an atomic vapor, and wherein the atomic vapor has an optical property capable of being changed by the presence of an external magnetic field across the chamber.

2. The device of claim 1, wherein the device comprises a light source capable of transmitting light to the atomic vapor in the chamber.

3. The device of claim 2, wherein the at least one magnetometer comprises a light source capable of transmitting light to the atomic vapor.

4. The device of claim 1, wherein the device comprises a photo detector capable of detecting an optical property of the atomic vapor.

5. The device of claim 4, wherein the at least one magnetometer comprises a photo detector capable of detecting an optical property of the atomic vapor.

6. The device of claim 1, further comprising a magnet capable of producing an oscillating magnetic field across at least one of the chambers.

7. The device of claim 6, wherein the oscillating magnetic field is oriented at about 45° with respect to the light transmitted to the atomic vapor in the chamber.

8. The device of claim 2, wherein the substrate comprises components capable of processing or analyzing signals from the photo detector.

9. The device of claim 8, wherein the components comprise one or more of a control, display, amplifier, microprocessor, MEMS, and integrated circuit.

10. The device of claim 1, wherein the array of optical magnetometers is placed on a surface of the substrate.

11. The device of claim 10, wherein the array of optical magnetometers is capable of being placed on or near a human's chest and that the device can detect magnetic fields of the human's heart.

12. The device of claim 11, wherein the surface of the substrate is flat, curved, or a combination thereof and wherein the magnetometers are capable of covering at least a portion of a human's chest.

13. The device of claim 10, wherein the array of optical magnetometers is capable of being placed on or near a human's head and can that the device detect magnetic fields of the human's brain.

14. The device of claim 13, wherein the surface of the substrate has a curved or helmet-like shape and wherein the array of optical magnetometers are capable of covering at least a portion of a human's head.

15. The device of claim 1, wherein at least a portion of the magnetometers are independently scalar or vector magnetometers.

16. The device of claim 1, the array of optical magnetometers is capable of providing data for generating a two-dimensional or three-dimensional mapping of the external magnetic field.

17. The device of claim 1, wherein at least a portion of the magnetometers further comprise a polarizer, a quarter plate, a filter, or a combination thereof.

18. The device of claim 1, wherein the container independently comprises silicon, glass, a polymer, or a combination thereof.

19. The device of claim 18, wherein the container comprises a combination of silicon and glass.

20. The device of claim 1, wherein the container and the chamber independently have a cylindrical, cubic, or cuboidal shape.

21. The device of claim 1, wherein the container has an overall dimension of from about 0.1 mm to about 10 cm.

22. The device of claim 1, wherein the container has an overall dimension of from about 1.0 mm to about 5.0 cm.

23. The device of claim 1, wherein the chamber has a volume of from about 100 (μm)³ to about 1.0 (cm)³.

24. The device of claim 23, wherein the chamber has a volume of from about 1000 (μm)³ to about 10 (mm)³.

25. The device of claim 1, wherein the atomic vapor comprises lithium (Li), sodium (Na), potassium (K), rubidium (Rb), cesium (Cs), and francium (Fr), or a combination thereof.

26. The device of claim 1, wherein the chamber is also filed with a buffer gas.

27. The device of claim 26, wherein the buffer gas comprises nitrogen, helium, neon, argon, krypton, xenon, radon, or a combination thereof.

28. The device of claim 2, wherein the light source comprises a laser.

29. The device of claim 28, wherein the laser is a vertical-cavity surface-emitting laser (VCSEL).

30. The device of claim 2, wherein the light source is capable of transmitting a circularly polarized laser beam to the atomic vapor.

31. The device of claim 2, wherein the light source comprises a beam splitter.

32. The device of claim 4, wherein the photo detector is a photodiode.

33. The device of claim 1, wherein the presence of an external magnetic field across each of at least a portion of the chambers is capable of changing the optical properties of the atomic vapors in each of the at least a portion of the chambers.

34. The device of claim 1, wherein the external magnetic field has a magnetic flux density of from about 10⁻¹⁶ Tesla (T) to about 10⁻⁹ T.

35. A method comprising:
providing a substrate; and
fabricating an array of optical magnetometers on a surface of the substrate;
wherein at least one of the magnetometers comprises a container having a chamber filled with an atomic vapor, and wherein the atomic vapor has an optical property capable of being changed by the presence of an external magnetic field across the chamber.

36. The method of claim 35, wherein the device comprises a light source capable of transmitting light to the atomic vapor in the chamber.

37. The method of claim 35, wherein the device comprises a photo detector capable of detecting an optical property of the atomic vapor.

38. The method of claim 35, further comprising providing a magnet capable of producing an oscillating magnetic field across at least one of the chambers.

39. The method of claim 38, wherein the oscillating magnetic field is oriented at about 45° with respect to the light transmitted to the atomic vapor in the chamber.

40. The method of claim 36, wherein the substrate comprises components capable of processing or analyzing signals from the photo detector.

41. The method of claim 40, wherein the components comprise one or more of a control, display, amplifier, microprocessor, MEMS, and integrated circuit.

42. The method of claim 35, wherein the array of optical magnetometers is fabricated on a surface of the substrate.

43. The method of claim 42, wherein the array of optical magnetometers is capable of being placed on or near a human's chest and that the device can detect magnetic fields of the human's heart.

44. The method of claim 42, wherein the array of optical magnetometers is capable of being placed on or near a human's head and can that the device detect magnetic fields of the human's brain.

45. The method of claim 35, wherein the container comprises silicon, glass, a polymer, or a combination thereof.

46. The method of claim 45, wherein the container comprises a combination of silicon and glass.

47. The method of claim 35, wherein the container and the chamber independently have a cylindrical, cubic, or cuboidal shape.

48. The method of claim 35, wherein the atomic vapor comprises lithium (Li), sodium (Na), potassium (K), rubidium (Rb), cesium (Cs), and francium (Fr), or a combination thereof.

49. The method of claim 35, wherein the chamber is also filed with a buffer gas.

50. The method of claim 35, wherein the photo detector is a photodiode.

51. The method of claim 35, wherein the presence of an external magnetic field across each of at least a portion of the chambers is capable of changing the optical properties of the atomic vapor in each of the at least a portion of the chambers.

52. A method comprising:
providing a device, the device comprising a substrate and an array of optical magnetometers placed on the substrate;
placing the device within an external magnetic field; and
detecting the external magnetic field by simultaneously using at least a portion of the array of optical magnetometers.

53. The method of claim 52, wherein each of the at least a portion of the array of optical magnetometers comprises:
a container having a chamber filled with an atomic vapor;
a light source capable of transmitting light to the atomic vapor in the chamber; and
a photo detector capable of detecting an optical property of the atomic vapor in the chamber.

54. The method of claim 53, further comprising providing an oscillating magnetic field across at least one of the chambers.

55. The method of claim 53, wherein the placing of the device within an external magnetic field comprises adjusting the location and orientation of the device.

56. The method of claim 52, wherein the detecting of the external magnetic field comprises detecting each distinct portion of the external magnetic field using a distinct magnetometer.

57. The method of claim 52, wherein each of at least a portion of the magnetometers is independently a scalar or vector magnetometer.

58. The method of claim 52, further comprising generating a two-dimensional or three-dimensional mapping of the external magnetic field.

59. The method of claim 52, wherein the method is carried out in a magnetically shield environment.

60. The method of claim 52, wherein the placing of the device within an external magnetic field comprises placing the device on or near a human's chest.

61. The method of claim 60, further comprising generating a magnetocardiogram (MCG) of the human.

62. The method of claim 52, wherein the placing of the device within an external magnetic field comprises placing the device on or near a human's head.

63. The method of claim 62, further comprising generating a magnetoencephalogram (MEG) of the human.
